(19) United States
(12) Patent Application Publication
(10) Pub. No.: US 2012/0130460 A1
(43) Pub. Date: May 24, 2012

(54) HYBRID IMPLANTABLE LEAD ASSEMBLY

(75) Inventors: Phong D. Doan, Stevenson Ranch, CA (US); Xiaoyi Min, Thousand Oaks, CA (US)

(73) Assignee: PACESETTER, INC., Sylmar, CA (US)

(21) Appl. No.: 12/951,805

(22) Filed: Nov. 22, 2010

(51) Int. Cl. A61N 1/05

(52) U.S. Cl. 607/116

(57) ABSTRACT

A hybrid implantable lead assembly includes a lead body, distal, proximal, and intermediate electrodes, coiled inductive elements, and an inductive circuit. The proximal and intermediate electrodes are disposed on the lead body between the distal electrode and a proximal end of the lead body. The proximal and intermediate electrodes are electrically connected with first and second pathways to sense electrical activity and/or deliver stimulus pulses. The first and second coiled inductive elements are electrically connected to the proximal and intermediate electrodes, respectively. The inductive circuit is electrically connected to the distal electrode. The first coiled inductive element and/or the second coiled inductive element has a first type of inductor structure and the inductive circuit has a different, second type of inductor structure that prevent magnetically induced electric current from flowing to the electrodes.

### FIELD OF THE INVENTION

One or more embodiments of the subject matter described herein generally relate to lead assemblies of implantable medical devices that are compatible with magnetic resonance imaging (MRI) systems.

### BACKGROUND OF THE INVENTION

Some known implantable lead assemblies that are used with implantable pulse generators (such as neurostimulators, pacemakers, defibrillators, or implantable cardioverter defibrillators) are prone to heating and induced current when placed in the strong static, gradient, and/or radiofrequency (RF) magnetic fields of a magnetic resonance imaging (MRI) system. The heating and induced current are the result of the lead assemblies acting as antennas in the magnetic fields generated during a MRI scan. Heating and induced current in the lead assemblies may result in deterioration of stimulation thresholds or, in the context of a cardiac lead, even increase the risk of cardiac tissue damage and perforation.

Many patients with an implantable pulse generator and implanted lead assembly may require, or can benefit from, a MRI scan in the diagnosis or treatment of a medical condition. MRI modality allows for flow visualization, characterization of vulnerable plaque, non-invasive angiography, assessment of ischemia and tissue perfusion, and a host of other applications. The diagnosis and treatment options enhanced by MRI may continue to increase over time. For example, MRI scans have been proposed as a visualization mechanism for lead implantation procedures.

Some known lead assemblies include co-radial conductive coils that extend through the lead assemblies to two different electrodes. The coils are mutually inductive such that current flowing through a first coil creates an induced current in the second coil and current flowing through the second coil creates an induced current in the first coil. When the lead assembly having the co-radial coils is exposed to an external magnetic field, such as the magnetic field generated by an MRI system, the magnetic field may create current in the coils. The mutual inductance of the coils can reduce the flow of the magnetic field-generated current. For example, the magnetic field-generated current in the first coil can create an induced current in the second coil that reduces the magnetic field-generated current in the second coil. Similarly, the magnetic field-generated current in the second coil can create an induced current in the first coil that reduces the magnetic field-generated current in the first coil.

In known lead assemblies having two electrodes, the co-radial coils can reduce magnetic field-generated current such that the magnetic field-generated current may only slightly heat the electrodes coupled with the coils. However, as the number of electrodes in the lead assembly increases, the inclusion of co-radial coils to reduce magnetic field-generated current may be unable to prevent significant heating of the electrodes. For example, with multi-electrode lead assemblies, the coils may be unable to prevent significant heating of the distal electrodes. The temperature of the distal tip electrode in a quadripole lead assembly may increase by approximately 20 degrees Fahrenheit (or approximately 10 degrees Centigrade) when the lead assembly is exposed to magnetic fields generated by MRI systems, such as 1.5 or 3.0 Tesla external magnetic fields. Such an increase in temperature may cause damage to the cardiac tissue to which the electrodes are fixed or otherwise in contact.

### BRIEF SUMMARY OF THE INVENTION

A hybrid implantable lead assembly is disclosed herein. In one embodiment, the hybrid implantable lead assembly includes a lead body, a distal electrode, proximal and intermediate electrodes, first and second coiled inductive elements, and an inductive circuit. The lead body extends between a distal end and an opposite proximal end. The distal electrode is disposed on the lead body near the distal end. The distal electrode is conductively coupled with a conductor to at least one of sense electrical activity or deliver stimulus pulses. The proximal and intermediate electrodes are disposed on the lead body between the distal electrode and the proximal end of the lead body. The proximal and intermediate electrodes are electrically connected with first and second pathways, respectively, to at least one of sense electrical activity or deliver stimulus pulses. The first and second coiled inductive elements are electrically connected to the proximal and intermediate electrodes, respectively. The inductive circuit is electrically connected to the distal electrode. At least one of the first coiled inductive element or the second coiled inductive element has a first type of inductor structure and the inductive circuit has a different, second type of inductor structure that prevent magnetically induced electric current from flowing to the proximal electrode, the intermediate electrode, and the distal electrode.

In another embodiment, another hybrid implantable lead assembly is provided. The lead assembly includes a lead body, distal and proximal electrodes, a distal inductive circuit, and first and second coiled conductors. The lead body extends between a distal end and an opposite proximal end along a longitudinal axis. The distal electrode is disposed on the lead body near the distal end of the lead body and is coupled with an elongated conductor disposed in the lead body. The distal inductive circuit is disposed in the lead body and is conductively coupled with the distal electrode. The proximal electrode is disposed on the lead body between the distal end and the proximal end of the lead body. The first and second coiled conductors are helically wrapped around the longitudinal axis in the lead body. The first coiled conductor is conductively coupled with the proximal electrode. The distal inductive circuit prevents magnetically induced current from flowing through the elongated conductor to the distal electrode. The second coiled conductor prevents magnetically induced current from flowing through the first coiled conductor to the proximal electrode by inducing a canceling current in the first coiled conductor.

In another embodiment, another hybrid implantable lead assembly is provided. The lead assembly includes an elongated lead body, distal and proximal electrodes, a conductive coil, a distal inductor, and an elongated conductor. The lead body extends along a longitudinal axis between a distal end and an opposite proximal end. The distal electrode is located on the lead body proximate to the distal end and the proximal electrode is located on the lead body between the distal electrode and the proximal end. The conductive coil is disposed in the lead body and extends along the longitudinal axis to the proximal electrode. The distal inductor is disposed in the lead body and is conductively coupled with the distal electrode. The distal inductor is conductively decoupled from the first conductive coil. The elongated conductor is disposed in the lead body and extends along the longitudinal axis and conductively coupled with the distal inductor. In one aspect, the distal inductor includes at least one of a band pass filter or an inductive circuit.

While multiple embodiments are disclosed, still other embodiments of the described subject matter will become apparent to those skilled in the art from the following Detailed Description, which shows and describes illustrative embodiments of disclosed inventive subject matter. As will be realized, the inventive subject matter is capable of modifications in various aspects, all without departing from the spirit and scope of the described subject matter. Accordingly, the drawings and detailed description are to be regarded as illustrative in nature and not restrictive.

### BRIEF DESCRIPTION OF THE DRAWINGS

FIG. 1 is a perspective view of one embodiment of a medical implantable hybrid lead assembly joined to an implantable medical device.

FIG. 2 is a longitudinal cross-sectional view of one embodiment of the hybrid lead assembly shown in FIG. 1.

FIG. 3 is a longitudinal cross-sectional view of a portion of a proximal segment of the hybrid lead assembly shown in FIG. 1 in accordance with one embodiment.

FIG. 4 is a longitudinal cross-sectional view of an intermediate segment of the hybrid lead assembly shown in FIG. 1 in accordance with one embodiment.

FIG. 5 is a circuit diagram of one embodiment of an inductive circuit.

FIG. 6 is a longitudinal cross-sectional view of a distal segment of the hybrid lead assembly shown in FIG. 1 in accordance with one embodiment.

### DETAILED DESCRIPTION

One or more embodiments described herein provide an implantable medical lead assembly that includes multi-electrodes and a plurality of different types of inductive elements that block the flow of magnetically induced electric current in conductive pathways of the lead assembly. By different “types” of inductive elements, the inductive elements have different inductor structures. One inductor structure may include mutually inductive conductive coils while another, different inductor structure may include an inductive circuit. The electrodes are used to deliver stimulus pulses to and/or sense cardiac signals of the heart. The lead assembly may be referred to as a hybrid MRI-compatible lead assembly because the lead assembly includes at least two separate inductive elements or inductors that reduce or prevent the flow of induced current through at least two separate electrodes of the lead assembly. The use of different types of inductive elements or inductor structures for separate electrodes may reduce the temperature increase of the electrodes that would otherwise be caused by magnetically induced current in lead assemblies having multiple electrodes, such as more than two electrodes. For example, the inductive elements conductively coupled to the distal electrodes may have first inductive characteristics while the inductive elements conductively coupled to the proximal electrodes have second inductive characteristics, such that the distal electrodes do not increase in temperature more than the proximal electrodes when exposed to a common external magnetic field.

In one embodiment, one type of inductive element may be first and second helically wound conductive coils that are disposed near each other. The second conductive coil is coupled to an electrode, such as a ring electrode. When the lead assembly is exposed to a relatively strong magnetic field, such as an external magnetic field created by an MRI system, the magnetic field induces current in the first and second conductive coils. The first and second coils are mutually inductive such that the coils induce current in each other. The mutual inductance of the coils can reduce or prevent the flow of magnetically induced current through the first and second coils and through the electrodes that are coupled with the coils.

Another, different type of inductive element of the lead assembly may be an inductive circuit. For example, a band pass filter, LC circuit, or RLC circuit may be conductively coupled with another electrode, such as a tip electrode. The inductive circuit prevents or reduces the flow of induced current to the electrode when the lead assembly is exposed to relatively strong magnetic fields.

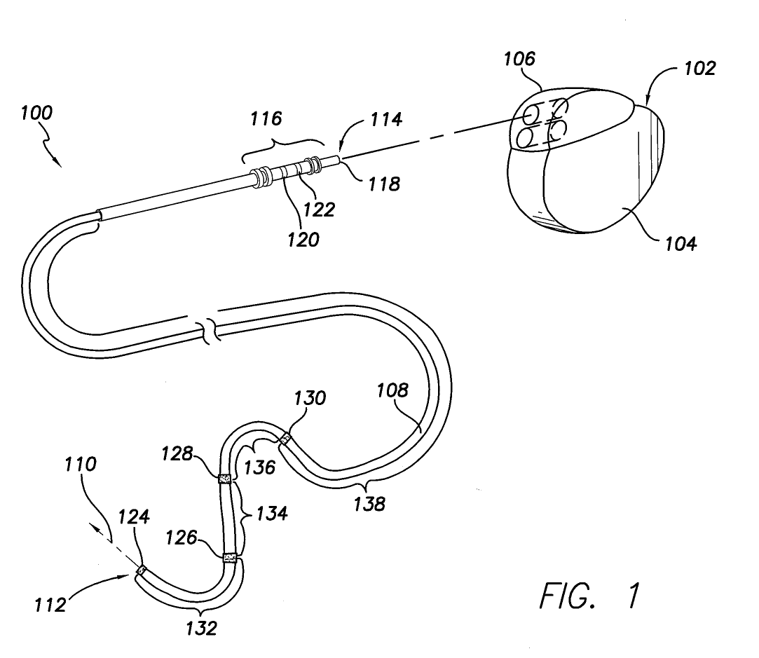

FIG. 1 is a perspective view of one embodiment of a medical implantable hybrid lead assembly 100 joined to an implantable medical device (IMD) 102. The hybrid lead assembly 100 is implanted into a heart of a patient to deliver stimulus pulses (such as pacing or defibrillation pulses) to the heart and/or sense cardiac signals of the heart. The IMD 102 generates the stimulus pulses and/or includes a processor to analyze the cardiac signals. The IMD 102 may be a pacemaker, defibrillator, implantable cardiac defibrillator (ICD), neurostimulator, and the like. The IMD 102 includes a housing 104 (also referred to as a “can”). The electrical components of the IMD 102 are disposed within the housing 104. The housing 104 includes a header 106 that receives the hybrid lead assembly 100.

The hybrid lead assembly 100 includes an elongated tubular body 108 extending along a longitudinal axis 110 from a distal end 112 to a proximal end 114. As shown in FIG. 1, the longitudinal axis 110 may include twists, turns, or undulations, and generally extend along a non-linear path. In one embodiment, the hybrid lead assembly 100 is a tachy lead, such as a Riata™ model lead manufactured by St. Jude Medical of St. Paul, Minn. In another embodiment, the hybrid lead assembly 100 is a different type, size, or model of a lead. For example, the hybrid lead assembly 100 may be a brady lead, such as 1888 Model lead manufactured by St. Jude Medical.

The proximal end 114 of the hybrid lead assembly 100 includes a header connector portion 116. The header connector portion 116 includes several conductive elements, such as a pin contact 118 and ring contacts 120, 122. The number and arrangement of conductive elements in the header connector portion 116 is provided merely as an example and is not intended to be limiting on all embodiments described herein. The header connector portion 116 is received in the header 106 of the IMD 102 such that the contacts 118, 120, 122 engage conductive terminals within the header 106 to connect the contacts 118, 120, 122 with the IMD 102.

One or more conductors provide separate electrically conductive pathways between the electrodes 124, 126, 128, 130 and the contacts 118, 120, 122. The conductors convey stimulus pulses from the IMD 102 to one or more of the electrodes 124, 126, 128, 130 and/or convey sensed cardiac signals from the electrodes 124, 126, 128, 130 to the IMD 102.

In the illustrated embodiment, the hybrid lead assembly 100 is a multi-electrode lead, such as a quadripole lead having four different electrodes 124, 126, 128, 130. The electrodes 124, 126, 128, 130 are used to deliver stimulus pulses to four different locations of the heart and/or to sense cardiac signals of the heart at four different locations of the heart. The electrodes 124, 126, 128, 130 include a distal tip electrode 124 located on the body 108 at or near the distal end 112 of the body 108. A proximal ring electrode 130 is located on the body 108 remote from the distal tip electrode 124. An intermediate distal electrode 126 and an intermediate proximal electrode 128 are located at intermediate points along the body 108 between the distal tip electrode 124 and the proximal ring electrode 130. The intermediate proximal electrode 128 is disposed proximal to the intermediate distal electrode 126 and the proximal ring electrode 130. The intermediate distal electrode 126 is located between the distal tip electrode 124 and the intermediate proximal electrode 128.

The distal tip electrode 124 may be positioned in contact with a free wall of the left ventricle of the heart in one embodiment. The electrodes 126, 128, 130 are ring electrodes that extend around the outer circumference of the body 108 and may be positioned in other locations of the left ventricle of the heart, such as locations between the free wall and the left atrium.

As shown in FIG. 1, the body 108 of the lead assembly 100 includes multiple successive segments 132, 134, 136, 138 that space the electrodes 124, 126, 128, 130 apart. Alternatively, the hybrid lead assembly 100 may include a different number of electrodes 124, 126, 128, 130 and/or a different number of segments 132, 134, 136, 138. In one embodiment, one or more segments 132, 134, 136, 138 includes more than a single electrode 124, 126, 128, or 130. The segments 132, 134, 136, 138 may have a common length or have different lengths thereby spacing successive electrodes 124, 126, 128, 130 corresponding common or different distances apart from one another.

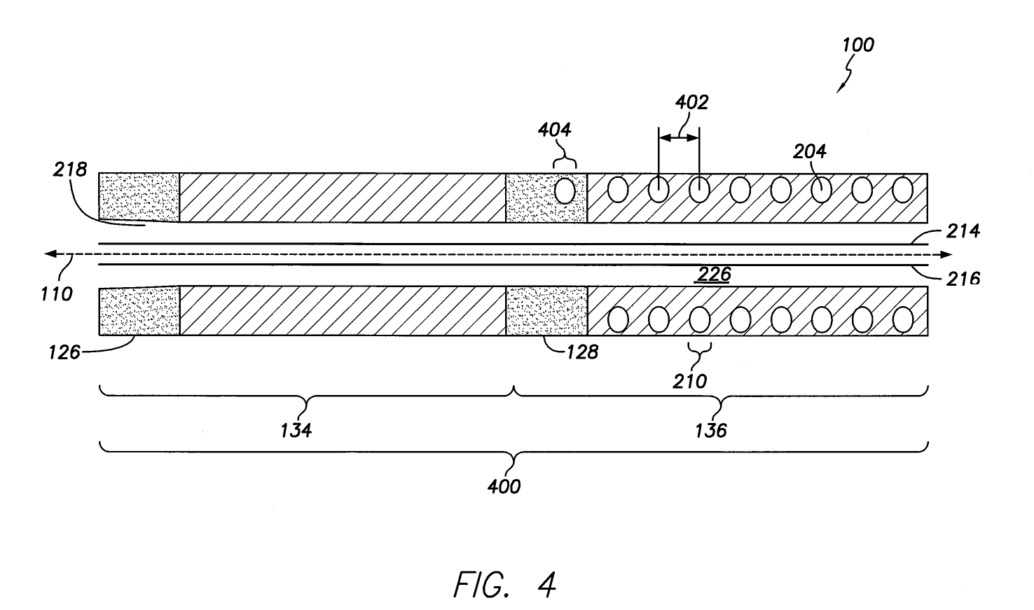

The segments 132, 134, 136, 138 represent a distal segment 132, an intermediate distal segment 134, an intermediate proximal segment 136, and a proximal segment 138. The intermediate distal and intermediate proximal segments 134, 136 may collectively be referred to as a cumulative intermediate segment 400 (shown in FIG. 4). As shown in FIG. 1, the proximal segment 138 may include a majority of the length of the body 108. Alternatively, the proximal segment 138 may include a smaller portion of the length of the body 108 when one or more electrodes are positioned further along the body 108 toward the header connector portion 116.

As described below, the segments 132, 134, 136, and 138 include separate and different inductive elements to reduce or prevent flow of magnetically induced current to the electrodes 124, 126, 128, and 130. For example, the distal and intermediate distal segments 132, 134 may include a band pass filter, an inductor-capacitor circuit (“LC circuit” or “tank circuit”), and/or a resistor-inductor-capacitor circuit (“RLC circuit”) that is conductively coupled with the distal and intermediate distal electrodes 124, 126. The intermediate proximal and proximal segments 136, 138 include conductive coils that are conductively coupled with the intermediate proximal and proximal electrodes 128, 130.

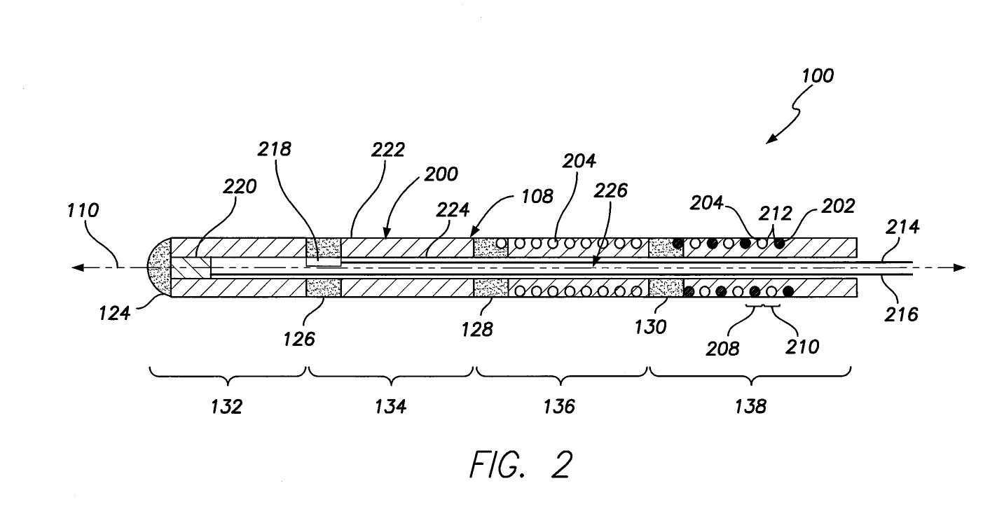

FIG. 2 is a longitudinal cross-sectional view of one embodiment of a distal portion of the hybrid lead assembly 100 shown in FIG. 1. FIG. 2 illustrates, in more detail, the successive segments 132, 134, 136, 138 and associated successive electrodes 124, 126, 128, 130. In the illustrated embodiment, only an outer portion of the proximal segment 138 is shown. The body 108 of the hybrid lead assembly 100 includes an outer tubing 200. The outer tubing 200 may be formed of a biocompatible electrical insulation material such as, for example, silicone rubber, silicone rubber polyurethane copolymer (“SPC”), polyurethane, or a Gore™ material. The outer tubing 200 extends between an exterior surface 222 and an interior surface 224. The electrodes 124, 126, 128, 130 are located within or over the outer tubing 200. The body 108 includes a central lumen 226 that is elongated along the longitudinal axis 110.

The hybrid lead assembly 100 includes co-radial coiled conductors 202, 204 extending through the outer tubing 200 along at least a portion of the length of the body 108. The coiled conductors 202, 204 include, or are formed from, wires or filars that are enclosed in electrically insulative jackets 212. The insulative jackets 212 prevent individual turns of the coiled conductors 202, 204 from directly electrically contacting adjacent turns of the same or a separate coiled conductor 202, 204 and forming a conductive pathway, shunt, or short therebetween. The insulative jackets 212 include, or are formed from, ethylene tetrafluoroethylene (ETFE), polytetrafluoroethylene (PTFE), perfluoroalkoxy copolymer resin (PFA), polyimide, polyurethane, silicone, or a combination of polyurethane and silicone, such as Optim™, and the like.

The coiled conductors 202, 204 are helically wrapped around or embedded within the outer tubing 200 of the hybrid lead assembly 100. The longitudinal cross-sectional view shown in FIG. 2 illustrates several turns 208, 210 of the coiled conductors 202, 204. The coiled conductor 202 spirals along the longitudinal axis 110 through at least a portion of the proximal segment 138. The coiled conductor 204 spirals along the longitudinal axis 110 through at least a portion of the proximal segment 138 and entirely spans across the intermediate proximal segment 136 in the illustrated embodiment.

The coiled conductor 202 is electrically connected to the proximal electrode 130 and the coiled conductor 204 is electrically connected to the intermediate proximal electrode 128 in the illustrated embodiment. The coiled conductor 204 terminates at the electrode 128 within the segment 136. The coiled conductor 202 terminates at the electrode 130 within the segment 138. The coiled conductor 202 forms part of the pathway along which stimulus pulses are delivered to the electrode 130 and sensed signals are received from the electrode 130. The coiled conductor 204 forms part of the pathway along which stimulus pulses are delivered to the electrode 128 and sensed signals are received from the electrode 128. Electric signals, such as cardiac signals, may be sensed by the electrodes 128, 130 and conveyed to the IMD 102 (shown in FIG. 1) via the conductive pathways provided by the coiled conductors 202, 204. Stimulus pulses, such as pacing pulses or spinal cord stimulation (SCS) pulses, may be supplied to the heart or nervous system of a patient via the coiled conductors 202, 204 and the electrodes 128, 130.

The coiled conductors 202, 204 may be sufficiently close to each other within at least the proximal segment 138 that the coiled conductors 202, 204 are mutually inductive. When the coiled conductors 202, 204 are mutually inductive, the flow of current through one coiled conductor 202 or 204 can induce electric current in the other coiled conductor 202 or 204. For example, the flow of electric current through the coiled conductor 202 may induce current in the coiled conductor 204. Similarly, the flow of electric current through the coiled conductor 204 may induce current in the coiled conductor 202.

The mutual inductance of the coiled conductors 202, 204 can suppress or prevent the flow of electric current through the elongated conductors coiled conductors 202, 204 that is induced by exposure of the lead assembly 100 to an external magnetic field (referred to herein as “magnetic field induced electric current” or “magnetic field induced current”). When the coiled conductors 202, 204 are exposed to relatively strong magnetic fields, such as magnetic fields generated by MRI systems, magnetic field induced current may be created in the coiled conductors 202, 204. The magnetic field induced currents may have the same or approximately the same magnitude but opposite polarities. The magnetic field induced current in the coiled conductor 202 can induce an electric current in the other coiled conductor 204 (referred to as a “conductor induced electric current,” “conductor induced current,” or a “canceling current”). Similarly, the magnetic field induced current in the coiled conductor 204 can create a conductor induced current in the coiled conductor 202. The conductor induced current in each of the coiled conductors 202, 204 may be approximately the same or the same magnitude but have an opposite polarity as the magnetic field induced currents in each of the coiled conductors 202, 204. The conductor induced current reduces or eliminates the magnetic field induced current in each of the coiled conductors 202, 204.

Elongated conductors 214, 216 are disposed within the body 108 and extend through the body 108 in directions that are parallel to the longitudinal axis 110. In the illustrated embodiment, the elongated conductors 214, 216 are disposed in the central lumen 226. The elongated conductors 214, 216 include, or are formed from, wires or filars that are enclosed in insulative jackets similar to the insulative jackets 212 of the coiled conductors 202, 204. The elongated conductors 214, 216 are linear or approximately linear bodies in the illustrated embodiment. For example, in contrast to the coiled conductors 202, 204, the elongated conductors 214, 216 are not wrapped or coiled around the longitudinal axis 110. Alternatively, the elongated conductors 214, 216 may be non-linear bodies, such as helically wrapped coiled conductors similar to the coiled conductors 202, 204.

The elongated conductors 214, 216 are conductively coupled with inductors 218, 220. The inductor 218 may be referred to as a proximal inductor and the inductor 220 may be referred to as a distal inductor due to the relative locations of the inductors 218, 220 in the lead body 108. The inductors 218, 220 are inductive elements or components disposed within the lead body 108. The inductors 218, 220 suppress or prevent the flow of magnetic field induced current through the elongated conductors 214, 216. As described below, the inductors 218, 220 may include one or more of electronic circuits that reduce or restrict the flow of induced electric current. By way of example only, the inductors 218, 220 may include band stop filters, inductor-capacitor (LC) circuits, resistor-inductor-capacitor (RLC) circuits, and/or integrated inductive circuits. The inductors 218, 220 can restrict or prevent the flow of magnetic field induced current, such as current that is induced by magnetic fields produced by MRI systems.

The inductors 218, 220 are conductively coupled with the electrodes 126, 124. For example, the inductor 218 is conductively coupled with the intermediate distal electrode 126 and the inductor 220 is conductively coupled with the distal electrode 124 in the illustrated embodiment. The inductors 218, 220 may be electrically connected in series with the elongated conductors 214, 216 and the electrodes 126, 124. The inductor 218 can electrically interconnect the elongated conductor 214 with the intermediate distal electrode 126 and the inductor 220 can electrically interconnect the elongated conductor 216 with the distal electrode 124.

In the illustrated embodiment, the lead assembly 100 includes different types of inductors for different electrodes to reduce or eliminate magnetic field induced current. As described above, the electrodes 128, 130 rely on mutual inductance between neighboring coiled conductors 202, 204 to reduce or eliminate magnetic field induced current while the electrodes 124, 126 rely on an additional inductive element or component to reduce or eliminate magnetic field induced current. For example, the electrodes 128, 130 use the same conductive components that are used to convey electric signals (such as cardiac signals) and/or deliver stimulus pulses through the electrodes 128, 130, while the electrodes 124, 126 use an additional inductive component, such as a band pass filter, LC circuit, RLC circuit, and the like, that is added to the conductive pathway through which electric signals are conveyed and/or stimulus pulses are delivered.

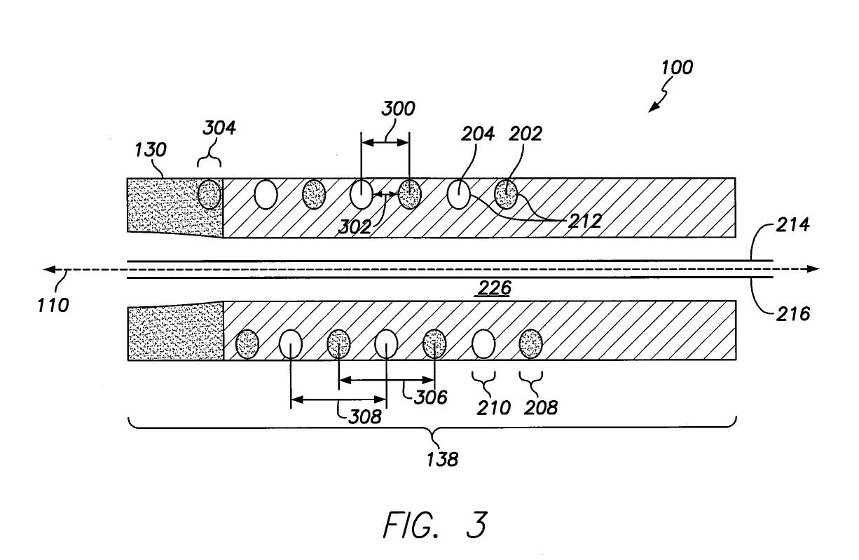

FIG. 3 is a longitudinal cross-sectional view of a portion of the proximal segment 138 of the hybrid lead assembly 100 in accordance with one embodiment. The coiled conductors 202, 204 and respective insulative jackets 212 are spaced apart from each other. For example, an inter-coil pitch dimension 300 is sufficiently large that an axial separation gap 302 exists between neighboring turns 208, 210 of the coiled conductors 202, 204. The inter-coil pitch dimension 300 is measured in directions along or parallel to the longitudinal axis 110 between common or similar points of the coiled conductors 202, 204. In the illustrated embodiment, the inter-coil pitch dimension 300 is measured between radial centers of the coiled conductors 202, 204. Alternatively, the inter-coil pitch dimension 300 may be small enough that the insulative jackets 212 of the turns 208 of the coiled conductor 202 engage the insulative jackets 212 of the turns 210 of the coiled conductor 204.

Changing the inter-coil pitch dimension 300 can vary a mutual inductance characteristic of the coiled conductors 202, 204. The mutual inductance characteristic represents the amount or magnitude of current that is induced in one of the coiled conductors 202, 204 by current flowing through the other of the coiled conductors 202, 204. For example, as the inter-coil pitch dimension 300 and/or the separation gap 302 between the turns 208, 210 of the coiled conductors 202, 204 increases or lengthens, less current is induced in the coiled conductor 204 by current flowing through the coiled conductor 202. Similarly, less current is induced in the coiled conductor 202 by current flowing through the coiled conductor 204. On the other hand, as the inter-coil pitch dimension 300 and/or the separation gap 302 between the turns 208, 210 of the coiled conductors 202, 204 decreases or shortens, more current is induced in the coiled conductor 204 by current flowing through the coiled conductor 202 and more current is induced in the coiled conductor 202 by current flowing through the coiled conductor 204.

The mutual inductance characteristic of the coiled conductors 202, 204 can be inversely related to the inter-coil pitch dimension 300 and/or the separation gap 302. When the inter-coil pitch dimension 300 or separation gap 302 increases in length, then the mutual inductance characteristic of the coiled conductors 202, 204 decreases. Conversely, when the inter-coil pitch dimension 300 or separation gap 302 decreases in length, the mutual inductance characteristic of the coiled conductors 202, 204 increases.

Sequential turns 208 of the coiled conductor 202 are axially separated from each other by a first intra-coil pitch dimension 306. For example, common points, such as the radial centers, of sequential turns 208 of the coiled conductor 202 are separated from each other by a distance measured along or parallel to the longitudinal axis 110 and referred to as the first intra-coil pitch dimension 306. Similarly, sequential turns 210 of the coiled conductor 204 are axially separated from each other by a second intra-coil pitch dimension 308. In one embodiment, the first and second intra-coil pitch dimensions 306, 308 are equivalent or approximately equivalent. Alternatively, the first and second intra-coil pitch dimensions 306, 308 may be different. In the illustrated embodiment, each of the first and second intra-coil pitch dimensions 306, 308 is approximately twice as long as the inter-coil pitch dimension 300.

As shown in FIG. 3, the elongated conductors 214, 216 extend through the proximal segment 138 of the lead assembly 100 without contacting or being conductively coupled to the electrode 130. For example, the elongated conductors 214, 216 may extend through the central lumen 226 without engaging the electrode 130. The elongated conductors 214, 216 may be enclosed in insulative jackets similar to the insulative jackets 212 such that the elongated conductors 214, 216 do not establish a conductive pathway between the elongated conductors 214, 216 if the insulative jackets of the elongated conductors 214, 216 engage each other. The insulative jackets of the elongated conductors 214, 216 also may prevent the elongated conductors 214, 216 from establishing a conductive pathway between the elongated conductors 214, 216 and the electrode 130 when the elongated conductors 214, 216 pass through the center of the electrode 130 within the central lumen 226.

The coiled conductor 202 extends into the electrode 130. For example, an end turn 304 of the coiled conductor 202 may be located within the conductive body of the electrode 130, as shown in FIG. 3. The insulative jacket of the portion of the coiled conductor 202 that is disposed within the electrode 130 may be removed to expose the coiled conductor 202. The exposure of the coiled conductor 202 within the electrode 130 conductively couples the coiled conductor 202 with the electrode 130. Alternatively, the coiled conductor 202 may be crimped to the electrode 130 or a conductive body, such as solder, may be conductively coupled to both the electrode 130 and the coiled conductor 202 to electrically join the coiled conductor 202 with the electrode 130.

Cardiac signals sensed by the electrode 130 can be conducted through the electrode 130 to the coiled conductor 202. The coiled conductor 202 conveys the cardiac signals to the IMD 102 (shown in FIG. 1). The IMD 102 can transmit electric stimulus pulses along the coiled conductor 202. The stimulus pulses are conducted to the electrode 130 where the stimulus pulses are applied to the heart of a patient.

The coiled conductor 204 is not conductively coupled with the electrode 130 in the illustrated embodiment. The coiled conductor 204 may not pass through the electrode 130 and instead may have increased separation between the turns 210 of the coiled conductor 204 that are on opposite sides of the electrode 130. For example, the electrode 130 may be positioned between sequential turns 210 of the coiled conductor 204. Alternatively, the coiled conductor 204 may extend through the electrode 130 with the insulative jacket of the coiled conductor 204 enclosing the coiled conductor 204 and preventing creation of a conductive pathway between the coiled conductor 204 and the electrode 130.

FIG. 4 is a longitudinal cross-sectional view of the intermediate segment 400 of the hybrid lead assembly 100 in accordance with one embodiment. The intermediate segment 400 includes the intermediate distal segment 134 joined with the intermediate proximal segment 136. As shown in FIG. 4, the intermediate distal segment 134 transitions into the intermediate proximal segment 136. The intermediate segment 400 is joined with and transitions into the proximal segment 138 (shown in FIG. 1) and is joined with and transitions into the distal segment 132 (shown in FIG. 1).

In the illustrated embodiment, the coiled conductor 202 (shown in FIG. 2) does not extend into the intermediate segment 400. For example, the coiled conductor 202 may extend into or otherwise be coupled with the electrode 130 (shown in FIG. 1) of the proximal segment 138 (shown in FIG. 1), but does not extend beyond the electrode 130 along the longitudinal axis 110 and into the intermediate segment 400. Alternatively, the coiled conductor 202 may extend into the intermediate segment 400. For example, the coiled conductor 202 can extend into the intermediate segment 400 such that the turns 208 (shown in FIG. 2) are disposed between sequential turns 210 of the coiled conductor 204, as shown in FIG. 3.

The coiled conductor 204 extends into the intermediate segment 400 by helically wrapping around the longitudinal axis 110 through at least a portion of the intermediate segment 400. In the illustrated embodiment, the coiled conductor 204 extends along the length of the lead assembly 100 to the electrode 128 but does not extend beyond the electrode 128. Alternatively, the coiled conductor 204 may extend along the longitudinal axis 110 beyond the electrode 128. For example, the coiled conductor 204 may continue to helically encircle the longitudinal axis 110 beyond the electrode 128 and toward the electrode 126.

Sequential turns 210 of the coiled conductor 204 are axially separated from each other by a third intra-coil pitch dimension 402. For example, common points, such as the radial centers, of sequential turns 210 of the coiled conductor 204 are separated from each other by a distance measured along or parallel to the longitudinal axis 110 and referred to as the third intra-coil pitch dimension 402. In the illustrated embodiment, the third intra-coil pitch dimension 402 is shorter than the second intra-coil pitch dimension 308 (shown in FIG. 3). The third intra-coil pitch dimension 402 may be shorter if the coiled conductor 204 is more tightly wrapped around the longitudinal axis 110 in the intermediate segment 400 than in the proximal segment 138 (shown in FIG. 1).

The coiled conductor 204 extends into the electrode 128. For example, an end turn 404 of the coiled conductor 204 may be located within the conductive body of the electrode 128, as shown in FIG. 4. The insulative jacket of the portion of the coiled conductor 204 that is disposed within the electrode 128 may be removed to expose the coiled conductor 204. The exposure of the coiled conductor 204 within the electrode 128 conductively couples the coiled conductor 204 with the electrode 128. Alternatively, the coiled conductor 204 may be crimped to the electrode 128 or a conductive body, such as solder, may be conductively coupled to both the electrode 128 and the coiled conductor 204 to electrically join the coiled conductor 204 with the electrode 128.

Cardiac signals sensed by the electrode 128 can be conducted through the electrode 128 to the coiled conductor 204. The coiled conductor 204 conveys the cardiac signals to the IMD 102 (shown in FIG. 1). The IMD 102 can transmit electric stimulus pulses along the coiled conductor 204. The stimulus pulses are conducted to the electrode 128 where the stimulus pulses are applied to the heart of a patient.

The elongated conductors 214, 216 extend through the intermediate proximal segment 136 of the lead assembly 100 without contacting or being conductively coupled to the electrode 128. For example, the elongated conductors 214, 216 may extend through the central lumen 226 without engaging the electrode 128. The elongated conductors 214, 216 also extend through the intermediate distal segment 134. As shown in FIG. 4, the elongated conductors 214, 216 extend through the central lumen 226 in the intermediate distal segment 134. The elongated conductor 214 is conductively coupled with the proximal inductor 218. The elongated conductor 216 is electrically separate from the proximal inductor 218 and the electrode 126 in that the elongated conductor 216 is not conductively coupled with the electrode 126.

As described above, the proximal inductor 218 is an inductive element or component that suppresses or prevents the flow of magnetic field induced current through the elongated conductor 214. The proximal inductor 218 is conductively coupled with the electrode 126 and conductively couples the elongated conductor 214 with the electrode 126. Alternatively, the proximal inductor 218 may be disposed in another location such that the proximal inductor 218 is conductively coupled with the elongated conductor 214 but is not disposed in series with the electrode 126 and the elongated conductor 214 in a location between the electrode 126 and the elongated conductor 214.

In one embodiment, the proximal inductor 218 is a different type of inductor than the coiled conductors 202, 204 (shown in FIG. 2). As described above, the coiled conductors 202, 204 suppress flow of induced current due to mutual inductance of the coiled conductors 202, 204. In contrast, the proximal inductor 218 may not rely on mutual inductance between the proximal inductor 218 and another conductive component that is used to convey cardiac signals and/or stimulus pulses. By way of example only, the proximal inductor 218 may include an inductive circuit that reduces or prevents the flow of induced current having a frequency within a frequency band of the proximal inductor 218. A “frequency band” is a range of frequencies that extend between and include lower and upper frequency thresholds.

In one embodiment, the proximal inductor 218 includes a band pass filter that removes or prevents the flow of current having a frequency within the frequency band of the band pass filter. For example, the proximal inductor 218 can be a band pass filter that prevents flow of current having a frequency that exceeds a lower frequency threshold and is no greater than an upper frequency threshold of the band pass filter. Alternatively, the proximal inductor 218 may blow flow of induced current that is greater than a lower frequency threshold without regard to an upper frequency threshold.

The lower frequency threshold of the proximal inductor 218 may be based on or associated with different MRI systems. By way of example only, an MRI system that generates a 1.5 Tesla magnetic field may induce a current in the elongated conductor 214 of approximately 64 MHz, an MRI system that generates 3.0 Tesla magnetic fields may induce currents of approximately 128 MHz, and so on. The lower frequency threshold may be set to block flow of induced current from one or more of these MRI systems. For example, the lower frequency threshold may be set to block current induced from 1.0 Tesla, 1.5 Tesla, 3.0 Tesla magnetic fields, and the like.

Alternatively, the proximal inductor 218 may include or be embodied in an electronic circuit, such as a tank circuit, an LC circuit, and/or an RLC circuit. Such a circuit may block the flow of induced current having a frequency that exceeds a lower threshold frequency and/or falls within a frequency band of the circuit.

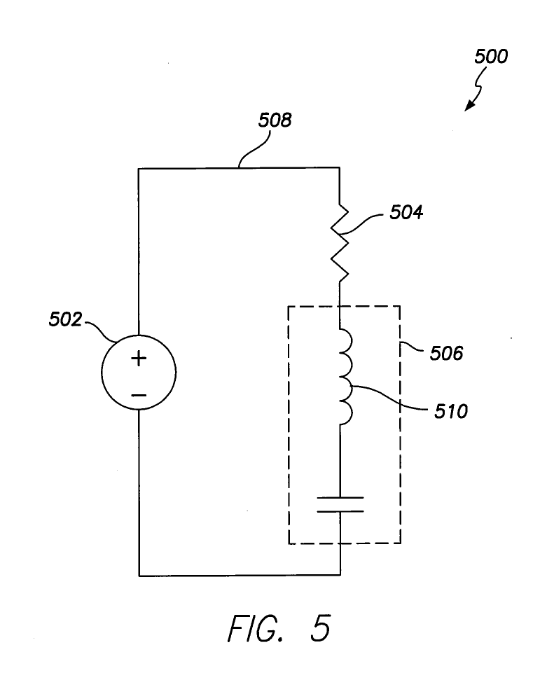

FIG. 5 is a circuit diagram of one embodiment of an inductive circuit 500. The inductive circuit 500 represents an electronic circuit that blocks flow of induced current that is created by an external magnetic field, such as a magnetic field generated by an MRI system. The circuit 500 includes a power source 502 coupled with a resistor 504 and an inductor-capacitor combination 506 by a conductive pathway 508. Additional, fewer, or different components of the circuit 500 may be provided in another embodiment. The circuit 500 represents the tank circuit, LC circuit, or RLC circuit of one or more of the inductors 218, 220 (shown in FIG. 2) in one embodiment.

The circuit 500 is disposed within the lead body 108 (shown in FIG. 1) and/or the IMD 102 (shown in FIG. 1). For example, the power source 502 may represent a source of electric current that is internal to the IMD 102, such as a battery. The conductive pathway 508 may represent a conductive wire, filar, bus, or other conductive body, such as the elongated conductor 214 (shown in FIG. 2). The resistor 504 may represent the electrode 126 (shown in FIG. 1) that is conductively coupled with the IMD 102 by the elongated conductor 214. The inductor-capacitor combination 506 may represent the proximal inductor 218 (shown in FIG. 2).

The inductor-capacitor combination 506 includes an inductive element 510 and a capacitive element 512. The inductive element 510 may be an electrical component that at least partially stores energy of electric current passing through the inductive element 510. The inductive element 510 can be provided as one or more inductors, such as a spiral inductor printed on a circuit board. Alternatively, the inductive element 510 may be another type of inductor capable of fitting within the lead body 108 (shown in FIG. 1).

The inductive element 510 stores energy of induced electric current passing through the conductive pathway 508 in a magnetic field generated by the inductive element 510. The inductive element 510 may store the energy of current having a frequency that is within a frequency range of the inductive element 510. For example, the inductive element 510 may permit current transmitted along the conductive pathway 508 at a frequency that is lower than a lower frequency threshold of the inductive element 510 or at a frequency that exceeds an upper frequency threshold of the inductive element 510 to pass through the inductive element 510 without being stored as energy. Conversely, electric current having a frequency within the frequency range, or between the lower and upper frequency thresholds of the inductive element 510, is at least partially temporarily stored in the inductive element 510 and prevented from flowing through the conductive pathway 508.

The frequency range of the inductive element 510 may be tuned to the frequencies of electric current that is induced in the conductive pathway 508 by exposure of the circuit 500 to MRI systems. For example, exposure of the circuit 500 to a 1.5 Tesla magnetic field created by an MRI system may induce current having a frequency of approximately 64 MHz in the conductive pathway 508. Exposure of the circuit 500 to a 3.0 Tesla magnetic field may induce current having a frequency of approximately 128 MHz. The frequency range of the inductive element 510 can be established to include one or more of these induced current frequencies.

The capacitive element 512 may be an electrical component that at least partially stores energy of electric current passing through the capacitive element 512. The capacitive element 512 can be provided as one or more capacitors, such as a plurality of spaced apart conductive bodies or plates separated by a dielectric. Alternatively, the capacitive element 512 may be another type of capacitor capable of fitting within the lead body 108 (shown in FIG. 1). The capacitive element 512 builds up electric charge from induced current flowing through the circuit 500. The built up electric charge creates an electric field within the capacitive element 512. At least some of the energy of the induced current is stored in this electric field.

In operation, electric current is induced in the circuit 500 when the circuit 500 is exposed to a relatively strong magnetic field. Energy of the induced electric current is stored between the inductive and capacitive elements 510, 512 and prevented from flowing through the conductive pathway 508 to the resistor 504 (such as an electrode). The induced electric current may flow, or vibrate, back and forth between the inductive and capacitive elements 510, 512 at a resonant frequency. For example, induced electric current can flow to the inductive element 510, where the current is at least partially stored by creating a magnetic field around or near the inductive element 510. At least some of the energy of the current that is stored in the magnetic field created by the inductive element 510 returns to the conductive pathway 508 as electric current that flows to the capacitive element 512. The current is at least partially stored in the capacitive element 512, but may again return to current that flows through the conductive pathway 508 to the inductive element 510. The back-and-forth oscillation of the induced current may occur at the resonant frequency.

The induced current oscillates back and forth between the inductive and capacitive elements 510, 512 and is prevented from flowing to the resistor 504, or the amount of current that flows to the resistor 504 is reduced. Internal resistance of the circuit 508 may eventually consume or deplete the energy of the induced current after the circuit 500 is no longer exposed to the external magnetic field.

The inductive characteristics of the inductive element 510 and the capacitive characteristics of the capacitive element 512 may be set or established based on the external magnetic fields to which the circuit 500 is exposed. For example, the inductance of the inductive element 510 and/or the capacitance of the capacitive element 512 may be established in order to prevent induced current generated by exposure of the circuit 500 to a 1.5 Tesla, 3.0 Tesla, and so on, external magnetic field of an MRI system. In one embodiment, the inductive element 510 has an inductance of approximately 270 nanoHenries and the capacitive element 512 has a capacitance of approximately 22 picoFarads. However, other values of the inductance and/or capacitance may be used.

Returning to the discussion of the intermediate segment 400 shown in FIG. 4, the proximal inductor 218 includes the circuit 500 (shown in FIG. 5), with the circuit 500 conductively coupled with the elongated conductor 214. For example, the conductive pathway 508 (shown in FIG. 5) of the circuit 500 may include part of, or be conductively coupled with, the elongated conductor 214. When electric current is induced in the elongated conductor 214, the circuit 500 of the proximal inductor 218 substantially reduces or prevents the induced current from flowing to and heating the electrode 218.

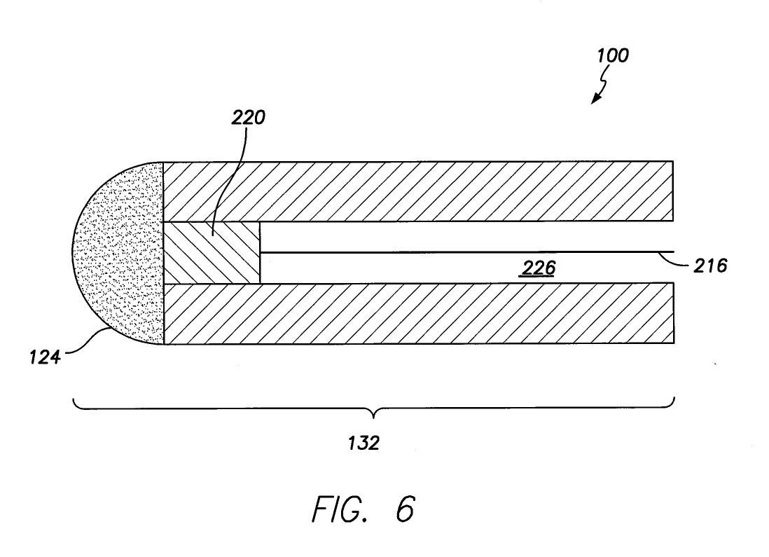

FIG. 6 is a longitudinal cross-sectional view of the distal segment 132 of the hybrid lead assembly 100 in accordance with one embodiment. The distal segment 132 is joined with and transitions into the intermediate segment 400 shown in FIG. 4. The elongated conductor 216 extends through the central lumen 226 of the distal segment 132 to the distal inductor 220. As described above, the distal inductor 220 is an inductive element or component that suppresses or prevents the flow of magnetic field induced current through the elongated conductor 216. In one embodiment, the distal inductor 220 includes one or more inductive circuits, such as the circuit 500 shown in FIG. 5.

The distal inductor 220 is conductively coupled with the tip electrode 124 and conductively couples the elongated conductor 216 with the tip electrode 124. Alternatively, the distal inductor 220 may be disposed in another location such that the distal inductor 220 is conductively coupled with the elongated conductor 216 but is not disposed in series with the tip electrode 124 and the elongated conductor 216 in a location between the tip electrode 124 and the elongated conductor 216.

The distal inductor 220 is a different type of inductor than the coiled conductors 202, 204 (shown in FIG. 2) in the illustrated embodiment. For example, instead of suppressing or preventing the flow of induced current through the elongated conductor 216 based on the mutual inductance between the distal inductor 220 and another electronic component, the distal inductor 220 may prevent or suppress flow of the induced current using a band pass filter similar to the proximal inductor 218 (shown in FIG. 2) and/or the circuit 500 (shown in FIG. 5), as described above. The band pass filter and/or the conductive pathway 508 (shown in FIG. 5) of the circuit 500 may be conductively coupled with the elongated conductor 216. When electric current is induced in the elongated conductor 216, the band pass filter and/or the circuit 500 of the distal inductor 218 substantially reduces or prevents the induced current from flowing to and heating the tip electrode 124.

One or more embodiments described herein provide a hybrid lead assembly having different types of inductors or inductive elements to substantially reduce, eliminate, or prevent the flow of electric current induced by an external magnetic field through filars to electrodes of the lead assembly. By “substantially reduce,” it is meant that the energy of the induced electric current is reduced such that the induced electric current does not cause heating of one or more of the electrodes of the lead assembly by more than 3, 5, 8, or 10 degrees Celsius. By “different types” of inductive elements, at least one embodiment described herein provides inductive elements that reduce or prevent the flow of induced current through different methods. One type of inductive element may rely on the mutual inductance between nearby conductive coils while another type of inductive element may rely on a circuit having inductive and capacitive elements.

It is to be understood that the subject matter described herein is not limited in its application to the details of construction and the arrangement of components set forth in the description herein or illustrated in the drawings hereof. The subject matter described herein is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings. Also, it is to be understood that phraseology and terminology used herein with reference to device or element orientation (such as, for example, terms like “central,” “upper,” “lower,” “front,” “rear,” “distal,” “proximal,” and the like) are only used to simplify description of one or more embodiments described herein, and do not alone indicate or imply that the device or element referred to must have a particular orientation. In addition, terms such as “outer” and “inner” are used herein for purposes of description and are not intended to indicate or imply relative importance or significance.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the presently described subject matter without departing from its scope. While the dimensions, types of materials and coatings described herein are intended to define the parameters of the disclosed subject matter, they are by no means limiting and are exemplary embodiments. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means—plus-function format and are not intended to be interpreted based on 35 U.S.C. §112, sixth paragraph, unless and until such claim limitations expressly use the phrase “means for” followed by a statement of function void of further structure.

Although the present invention has been described with reference to preferred embodiments, persons skilled in the art will recognize that changes may be made in form and detail without departing from the spirit and scope of the invention.

What is claimed is:

1. A hybrid implantable lead assembly comprising:
a lead body extending between a distal end and an opposite proximal end;
a distal electrode disposed on the lead body near the distal end, the distal electrode conductively coupled with a conductor to at least one of sense electrical activity or deliver stimulus pulses;
proximal and intermediate electrodes disposed on the lead body between the distal electrode and the proximal end of the lead body, the proximal and intermediate electrodes electrically connected with first and second pathways, respectively, to at least one of sense electrical activity or deliver stimulus pulses;
first and second coiled inductive elements electrically connected to the proximal and intermediate electrodes, respectively; and
an inductive circuit electrically connected to the distal electrode, wherein at least one of the first coiled inductive element or the second coiled inductive element has a first type of inductor structure and the inductive circuit has a different, second type of inductor structure that prevent magnetically induced electric current from flowing to the proximal electrode, the intermediate electrode, and the distal electrode.

2. The lead assembly of claim 1, wherein the first type of inductor structure of the first and second coiled inductive elements uses mutual inductance between the first and second coiled inductive elements to prevent flow of the magnetically induced electric current through at least one of the proximal electrode or the intermediate electrode.

3. The lead assembly of claim 1, wherein the first and second coiled inductive elements are co-radial conductive coils helically wrapped around a longitudinal axis of the lead body.

4. The lead assembly of claim 3, wherein the first coiled inductive element longitudinally extends to and terminates at the proximal electrode and the second coiled inductive element longitudinally extends to and terminates at the intermediate electrode.

5. The lead assembly of claim 1, wherein the second type of inductor structure of the inductive circuit includes a tank circuit to prevent flow of the magnetically induced electric current through the distal electrode.

6. The lead assembly of claim 1, wherein the second type of inductor structure of the inductive circuit includes a band pass filter to prevent flow of the magnetically induced electric current through the distal electrode.

7. The lead assembly of claim 1, further comprising a distal intermediate electrode disposed on the lead body between the distal electrode and the intermediate electrode, the distal intermediate electrode conductively coupled with a third inductive element that is a common type of inductor structure as the first or second inductor structure.

8. A hybrid implantable lead assembly comprising:
a lead body extending between a distal end and an opposite proximal end along a longitudinal axis;
a distal electrode disposed on the lead body near the distal end, the distal electrode conductively coupled with a conductor disposed in the lead body;
a distal inductive circuit disposed in the lead body and conductively coupled with the distal electrode;
a proximal electrode disposed on the lead body between the distal end and the proximal end of the lead body; and
first and second coiled conductors helically wrapped around the longitudinal axis in the lead body, the first coiled conductor conductively coupled with the proximal electrode, wherein the distal inductive circuit prevents magnetically induced current from flowing through the elongated conductor to the distal electrode, the second coiled conductor preventing magnetically induced current from flowing through the first coiled conductor to the proximal electrode by inducing a canceling current in the first coiled conductor.

9. The lead assembly of claim 8, wherein the first and second coiled conductors are co-radial coils.

10. The lead assembly of claim 8, wherein the distal inductive circuit includes at least one of a band pass filter or a tank circuit.

11. The lead assembly of claim 8, further including an intermediate electrode disposed on the lead body between the distal and proximal electrodes, the intermediate electrode conductively coupled with the second coiled conductor.

12. The lead assembly of claim 11, wherein each of the first and second coiled conductors prevents flow of the magnetically induced current to the proximal and intermediate electrodes by inducing the canceling current in the other of the first and second coiled conductors.

13. The lead assembly of claim 8, further comprising an intermediate electrode disposed on the lead body between the distal and proximal electrodes, the intermediate electrode conductively coupled with a proximal inductive circuit that prevents flow of the magnetically induced current to the intermediate electrode.

14. The lead assembly of claim 8, further comprising an intermediate distal electrode and an intermediate proximal electrode disposed on the lead body between the distal and proximal electrodes, the intermediate proximal electrode conductively coupled with the second coiled conductor, the intermediate distal electrode conductively coupled with a proximal inductive circuit.

15. A hybrid implantable lead assembly comprising:
an elongated lead body extending along a longitudinal axis between a distal end and an opposite proximal end;
a distal electrode located on the lead body proximate to the distal end;
a proximal electrode located on the lead body between the distal electrode and the proximal end;
a first conductive coil disposed in the lead body, the first conductive coil extending along the longitudinal axis to the proximal electrode;
a distal inductor disposed in the lead body and conductively coupled with the distal electrode, the distal inductor conductively decoupled from the first conductive coil; and
a first elongated conductor disposed in the lead body, the first elongated conductor extending along the longitudinal axis and conductively coupled with the distal inductor.

16. The lead assembly of claim 15, wherein the distal inductor includes at least one of a band pass filter or an inductive circuit.

17. The lead assembly of claim 15, wherein the distal inductor prevents flow of magnetically induced current through the first elongated conductor to the distal electrode.

18. The lead assembly of claim 15, further comprising: an intermediate distal electrode located on the lead body between the distal electrode and the proximal electrode; an intermediate inductor disposed in the lead body and conductively coupled with the intermediate distal electrode; and a second elongated conductor disposed in the lead body, the second elongated conductor extending along the longitudinal axis and conductively coupled with the intermediate inductor.

19. The lead assembly of claim 15, further comprising: an intermediate proximal electrode located on the lead body between the distal electrode and the proximal electrode; and a second conductive coil disposed in the lead body, the first conductor coil extending along the longitudinal axis to the intermediate proximal electrode.

20. The lead assembly of claim 19, wherein each of the first and second conductive coils prevent flow of magnetically induced current through the other of the first and second conductive coils to the proximal electrode and the intermediate proximal electrode.

21. The lead assembly of claim 19, wherein the first and second conductive coils are co-radial coils that are helically wrapped around the longitudinal axis.

22. The lead assembly of claim 19, wherein the lead body includes a distal segment, a proximal segment, and an intermediate segment extending from the distal segment to the proximal segment, the intermediate proximal electrode disposed in the intermediate segment, the proximal electrode located in the proximal segment, further wherein both the first and second conductive coils extend through the proximal segment while only the second conductive coil of the first and second conductive coils extends into the intermediate segment.

23. The lead assembly of claim 19, wherein a first pitch distance of neighboring turns in the second conductive coil between the proximal electrode and the proximal end of the lead body is longer than a second pitch distance of neighboring turns in the second conductive coil between the intermediate proximal electrode and the proximal electrode.

24. The lead assembly of claim 19, further comprising a lumen disposed within the lead body, the first elongated conductor extending through the lumen.

25. The lead assembly of claim 15, wherein the proximal end of the lead body include contacts configured to conductively couple the first elongated conductor and the first conductive coil with a medical device to at least one of deliver stimulus pulses or sense electrical activity of a heart using the distal and proximal electrodes.
